(19) United States
(12) Patent Application Publication
(10) Pub. No.: US 2026/0315381 A1
(43) Pub. Date: Oct. 15, 2026

(54) COLLAGEN AND CERAMIDE BEVERAGE COMPOSITIONS

(71) Applicant: Melaleuca, Inc., Idaho Falls, ID (US)

(72) Inventors: Jordan Joy, Idaho Falls, ID (US); Nasser A. Fredj, Idaho Falls, ID (US)

(21) Appl. No.: 19/633,394

(22) Filed: Mar. 30, 2026

Related U.S. Application Data
(60) Provisional application No. 63/780,876, filed on Mar. 31, 2025.

(51) Int. Cl. A61K 9/00; A23L 2/39; A23L 2/58; A23L 2/66; A61K 31/07; A61K 31/122; A61K 31/164; A61K 31/375; A61K 38/39; A61K 47/46

(52) U.S. Cl. CPC A61K 9/0095; A23L 2/39; A23L 2/58; A23L 2/66; A61K 31/07; A61K 31/122; A61K 31/164; A61K 31/375; A61K 38/39; A61K 47/46

(57) ABSTRACT

Beverage compositions containing bioactive collagen peptides and ceramides are provided herein, as are methods for using the beverage compositions to improve skin, joint, nail, and/or hair health.

### CROSS-REFERENCE TO RELATED APPLICATIONS

This application claims priority from U.S. Provisional Application Ser. No. 63/780,876, filed Mar. 31, 2025. The disclosure of the prior application is considered part of (and is incorporated by reference in) the disclosure of this application.

### TECHNICAL FIELD

This disclosure relates to beverage mixes containing bioactive collagen and ceramides, and to methods of making and using such beverage mixes.

### BACKGROUND

Skin is primarily composed of collagen proteins, which give skin its firmness and smoothness. However, the collagen content of skin declines with age and with UV exposure; age involves, in general, a progressive loss of function in the ability to make proteins, and UV exposure increases free radicals, which damage existing skin proteins such as collagen, reducing skin firmness and hydration.

Joints correspond to junctions between bones where articulation occurs. Cartilage, which is made from collagen proteins, is present in joints to protect the bones from wear. With advanced age, cartilage wears down and bones can begin to make contact, causing pain, reducing physical abilities, and compromising quality of life.

Consumers seek products that preserve their youth, including skin and joint health. Therefore, products that improve skin and joint health are highly desirable.

### SUMMARY

Provided herein are beverage compositions that include particular bioactive collagen peptides, as well as “standard” collagen peptides, ceramides, vitamin A, and vitamin C. The beverage compositions described herein can be ingested to improve the overall health of skin, joints, and/or hair. For example, the ceramides in the compositions can provide skin moisturization benefits to the compositions, in addition to the collagen-boosting benefits provided by the bioactive collagen peptides and the anti-oxidant benefits provided by the vitamins.

In a first aspect, this document features a powdered beverage composition. The composition can contain, consist essentially of, or consist of first collagen peptides, second collagen peptides, and ceramides, wherein the first collagen peptides have an average molecular weight of about 1 kDa to about 4 kDa, and wherein the second collagen peptides have an average molecular weight of about 2 kDa to about 5 kDa. The first collagen peptides can be present in the composition in an amount of from about 15 wt % to about 30 wt %. The second collagen peptides can be present in the composition in an amount of from about 35 wt % to about 55 wt %. The ceramides can be present in the composition in an amount of from about 0.1 wt % to about 1 wt %. The powdered beverage composition can further contain one or more vitamins. The one or more vitamins can include vitamin A palmitate. The vitamin A palmitate can be present in the composition in an amount of from about 0.01 wt % to about 0.1 wt %. The one or more vitamins can include ascorbic acid. The ascorbic acid can be present in the composition in an amount of from about 0.1 wt % to about 1 wt/o. The powdered beverage composition can further include astaxanthin. The astaxanthin can be present in the composition in an amount of from about 0.5 wt % to about 2.5 wt/o.

In another aspect, this document features a powdered beverage composition, where the powdered beverage composition contains, consists of, or consists essentially of collagen peptides, ceramides, and at least one antioxidant, wherein the collagen peptides have an average molecular weight of about 1 kDa to about 5 kDa. The collagen peptides can be present in the composition in an amount of from about 15 wt % to about 55 wt %. The ceramides can be present in the composition in an amount of from about 0.1 wt % to about 1 wt %. The at least one antioxidant can include one or more vitamins. The one or more vitamins can include vitamin A palmitate. The vitamin A palmitate can be present in the composition in an amount of from about 0.01 wt % to about 0.1 wt %. The one or more vitamins can include ascorbic acid. The ascorbic acid can be present in the composition in an amount of from about 0.1 wt % to about 1 wt %. The powdered beverage composition can further include astaxanthin. The astaxanthin can be present in the composition in an amount of from about 0.5 wt % to about 2.5 wt %.

In another aspect, this document features a powdered beverage composition, where the powdered beverage composition contains, consists essentially of, or consists of first collagen peptides, second collagen peptides, ceramides, and at least one antioxidant, wherein the first collagen peptides have an average molecular weight of about 1 kDa to about 4 kDa, wherein the second collagen peptides have an average molecular weight of about 2 kDa to about 5 kDa, and wherein the ceramides are present in the composition in an amount of from about 0.1 wt % to about 1 wt %. The first collagen peptides can be present in the composition in an amount of from about 15 wt % to about 30 wt %. The second collagen peptides can be present in the composition in an amount of from about 35 wt % to about 55 wt %. The at least one antioxidant can be selected from vitamin A and vitamin C. The vitamin A can be present in the composition in an amount of from about 0.01 wt % to about 0.1 wt %. The vitamin C can be present in the composition in an amount of from about 0.1 wt % to about 1 wt %. The powdered beverage composition can further include astaxanthin. The astaxanthin can be present in the composition in an amount of from about 0.5 wt % to about 2.5 wt %.

In still another aspect, this document features a powdered beverage composition, where the powdered beverage composition contains, consists essentially of, or consists of first collagen peptides, second collagen peptides, ceramides, and astaxanthin, wherein the first collagen peptides have an average molecular weight of about 1 kDa to about 4 kDa, and wherein the second collagen peptides have an average molecular weight of about 2 kDa to about 5 kDa, and wherein the ceramides are present in the composition in an amount of from about 0.1 wt % to about 1 wt %. The first collagen peptides can be present in the composition in an amount of from about 15 wt % to about 30 wt %. The second collagen peptides can be present in the composition in an amount of from about 35 wt % to about 55 wt/o. The ceramides can be present in the composition in an amount of from about 0.1 wt % to about 1 wt %. The astaxanthin can be present in the composition in an amount of from about 0.5 wt % to about 2.5 wt %. The powdered beverage composition can further contain one or more vitamins. The one or more vitamins can include vitamin A palmitate. The vitamin A palmitate can be present in the composition in an amount of from about 0.01 wt % to about 0.1 wt %. The one or more vitamins can include ascorbic acid. The ascorbic acid can be present in the composition in an amount of from about 0.1 wt % to about 1 wt %.

In yet another aspect, this document features a powdered beverage mix composition contained in a bulk package. The bulk package can contain a measuring scoop and the powdered beverage mix composition, wherein the measuring scoop is sized to scoop an amount of the powdered beverage mix composition suitable for blending with a liquid to form a beverage, wherein the amount of the powdered beverage mix composition is 10 to 20 g and contains, consists essentially of, or consists of: (a) from about 15 wt % to about 30 wt % of first collagen peptides, wherein the first collagen peptides have an average molecular weight of about 1 kDa to about 4 kDa; (b) from about 35 wt % to about 55 wt % of second collagen peptides, wherein the second collagen peptides have an average molecular weight of about 2 kDa to about 5 kDa; and (c) from about 0.1 wt % to about 0.5 wt % ceramides. The powdered beverage composition can further contain one or more vitamins. The one or more vitamins can include vitamin A palmitate. The vitamin A palmitate can be present in the composition in an amount of from about 0.01 wt % to about 0.1 wt %. The one or more vitamins can include ascorbic acid. The ascorbic acid can be present in the composition in an amount of from about 0.1 wt % to about 1 wt %. The powdered beverage composition can further include astaxanthin. The astaxanthin can be present in the composition in an amount of from about 0.5 wt % to about 2.5 wt %.

This document also features a method of improving skin, joint, and/or hair health. The method can include, or consist essentially of, solubilizing a powdered beverage composition described herein in an aqueous carrier, and ingesting an effective amount of the solubilized beverage composition, thereby improving skin, joint, and/or hair health. The aqueous carrier can be water. In some cases, the aqueous carrier can be coffee, cocoa, or tea.

In another aspect, this document features a method for making a beverage mix composition described herein. The method can include, or consist essentially of, combining first collagen peptides, second collagen peptides, and ceramides. The first collagen peptides can be present in the composition in an amount of from about 15 wt % to about 30 wt %. The second collagen peptides can be present in the composition in an amount of from about 35 wt % to about 55 wt %. The ceramides can be present in the composition in an amount of from about 0.1 wt % to about 1 wt %. The method can further comprise adding one or more vitamins to the composition. The one or more vitamins can include vitamin A palmitate. The vitamin A palmitate can be present in the composition in an amount of from about 0.01 wt % to about 0.1 wt %. The one or more vitamins can include ascorbic acid. The ascorbic acid can be present in the composition in an amount of from about 0.1 wt % to about 1 wt %. The composition can further comprise adding astaxanthin to the composition. The astaxanthin can be present in the composition in an amount of from about 0.5 wt % to about 2.5 wt %. The method can further include adding an aqueous carrier. The aqueous carrier can be water. In some cases, the aqueous carrier can be coffee, cocoa, or tea.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

This document provides beverage mix formulations containing biologically active collagen peptides that can increase endogenous collagen production site-specifically within the skin and joints. The beverage compositions provided herein also contain ceramides, which can increase skin hydration and provide other benefits. In addition, the beverage compositions provided herein can contain astaxanthin (which can provide anti-oxidant benefits), vitamin A, and/or vitamin C. The collagen, ceramide, and astaxanthin ingredients can provide benefits such as, without limitation, improved skin hydration, protection from photoaging, improved muscle recovery after exercise, supporting a healthy immune system, reducing skin redness, improving skin tone, reducing oxidation and DNA damage, decreasing the appearance of cellulite, improving skin elasticity, improving fingernail strength, reducing the appearance of fine lines and wrinkles, helping to rebuild cartilage, improving joint function, decreasing the occurrence of broken nails, reducing joint discomfort, strengthening hair, improving skin hydration, reducing transepidermal water loss, and supporting the skin's barrier function. The beverage compositions provided herein can be in the form of a solid (e.g., a powdered beverage mix) or a liquid (e.g., a powdered beverage mix dissolved or suspended in an ingestible fluid such as water or any suitable beverage).

### Collagen

The beverage compositions provided herein can contain any appropriate bioactive collagen peptides. In some cases, the beverage compositions provided herein can contain two or more different bioactive collagen peptide ingredients. For example, the beverage compositions provided herein can contain a first ingredient that includes bioactive collagen peptides having an average molecular weight of about 1 kDa to about 4 kDa. Without being bound by theory, such bioactive collagen peptides can increase collagen mRNA and stimulate fibroblast cells by at least two-fold more than generic collagen peptides. Bioactive collagen peptides having a molecular weight of about 1 kDa to about 4 kDa can be present in the beverage compositions provided herein in any appropriate amount. For example, from about 1 g to about 10 g (e.g., about 2 g to about 7.5 g, about 2.5 g to about 5 g, about 3 g to about 4 g, about 1 g to about 5 g about 1 g, about 2 g, about 2.5 g about 3 g, about 4 g about 5 g or about 10 g) of the first bioactive collagen peptides can be included in a beverage composition as described herein. In some cases, a beverage composition provided herein can contain about 10 wt % to about 40 wt % (e.g., about 15 wt % to about 30 wt %, about 20 wt % to about 25 wt %, about 15 wt %, about 20 wt %, about 23 wt %, about 25 wt %, about 27 wt %, or about 30 wt %) of bioactive collagen peptides having a molecular weight of about 1 kDa to about 4 kDa. The first bioactive collagen peptides can take any number of forms including, for example, a hydrolyzed powder. Those of skill in the art would be able to generate bioactive collagen peptides having an average molecular weight in the range of about 1 kDa to about 4 kDa using known methods.

In some cases, the beverage compositions provided herein can contain a second ingredient that also includes bioactive collagen peptides, where the bioactive collagen peptides in the second ingredient have an average molecular weight of about 2 kDa to about 5 kDa. Without being bound by theory, such bioactive collagen peptides can stimulate collagen production in chondrocytes at a rate that is twice that of other collagen peptides. Bioactive collagen peptides having a molecular weight of about 2 kDa to about 5 kDa can be present in the beverage compositions provided herein in any appropriate amount. For example, from about 2.5 g to about 20 g (e.g., about 2 g to about 10 g, about 3 g to about 15 g, about 5 g to about 10 g, about 6 g to about 8 g, about 3 g, about 5 g, about 10 g, about 15 g, or about 20 g) of the second bioactive collagen peptides can be included in a beverage composition as described herein. In some cases, a beverage composition provided herein can contain about 30 wt % to about 60 wt % (e.g., about 35 wt % to about 55 wt %, about 40 wt % to about 50 wt %, about 40 wt %, about 43 wt %, about 45 wt %, about 46 wt %, about 48 wt %, or about 50 wt %) of bioactive collagen peptides having a molecular weight of about 2 kDa to about 5 kDa. As above, the second bioactive collagen peptides can take any number of forms including, for example, a hydrolyzed powder. Those of skill in the art would be able to generate bioactive collagen peptides having an average molecular weight of about 2 kDa to about 5 kDa using known methods.

Without being bound by theory, it is believed that both the first bioactive collagen peptides and the second bioactive collagen peptides can bind to integrins on cell membranes and activate collagen production within the target cells, irrespective of the type of collagen (I, II, III, V, or otherwise) from which they are derived. Existing collagen products primarily use collagen peptides made from a non-specific enzymatic hydrolysis, which are not believed to independently improve skin health and anti-aging. On the other hand, the first and second bioactive collagen peptides in the beverage compositions provided herein can reduce wrinkles, cellulite, and joint discomfort in humans while increasing cartilage production in the joints. Moreover, the first bioactive collagen peptides (e.g., about 2 kDa bioactive collagen peptides) can improve skin health and reduce cellulite at a dose of about 2.5 g daily. Further, while a dose of generic collagen may be able to improve joint comfort to a certain extent, the same can be achieved with about half as much of the second bioactive collagen peptides (e.g., about 3 kDa bioactive collagen peptides). In addition, at some doses, the second bioactive collagen peptides also can increase cartilage regeneration.

In some cases, the beverage compositions provided herein also can contain a third ingredient that includes standard collagen peptides having an average molecular weight of about 1 kDa to about 5 kDa. In some cases, the collagen peptides in the third ingredient can be de-flavored so they do not affect the taste of the beverage composition. Exemplary standard collagen peptides include, without limitation, Peptiplus® and Peptiplus XB AGGL® (Gelita). It is noted, however, that a skilled artisan would be able to generate standard collagen peptides having an average molecular weight of about 1 kDa to about 5 kDa using known methods. Standard collagen peptides having a molecular weight of about 1 kDa to about 5 kDa can be present in the beverage compositions provided herein in any appropriate amount. For example, from 1 g to about 10 g (e.g., about 1 g to about 5 g, about 2 g to about 4 g, about 2.5 g to about 7.5 g, about 3 g to about 6 g, about 2 g, about 2.5 g, about 3 g, or about 5 g) of standard collagen peptides can be included in a beverage composition provided herein. In some cases, a beverage composition provided herein can contain about 10 wt % to about 40 wt % (e.g., about 15 wt % to about 30 wt %, about 20 wt % to about 25 wt %, about 15 wt %, about 20 wt %, about 23 wt %, about 26 wt %, about 28 wt %, or about 30 wt %) of standard collagen peptides.

The beverage compositions provided herein can include any appropriate total amount of collagen peptides. For example, the bioactive collagen peptides and the standard collagen peptides can be present in a beverage composition provided herein in a combined total amount from about 5 g to about 25 g (e.g., about 5 g to about 10 g, about 10 g to about 15 g, about 10 g to about 20 g, about 15 g to about 20 g, about 20 g to about 25 g, about 5 g, about 7.5 g, about 10 g, about 12.5 g, or about 15 g).

### Ceramides

The beverage compositions provided herein also contain ceramides. Ceramides are waxy lipid molecules that include sphingosine and a fatty acid linked by an amide bond (Formula I below). Ceramides are found in skin cells, and are important for retaining the skin's moisture. The beverage compositions provided herein can contain one or more (e.g., two, three, four, five, six, seven, eight, nine, ten, or more than ten) different types of ceramides, and/or complexes of ceramide molecules. In some cases, the ceramide molecules and/or complexes can include glucosyl-type ceramides, which have a glucose moiety in structures such as, without limitation, glucosyl-Cer(d18:2/16:0), Cer(d18:1/16:0), Cer(d18:1/20:0), and Cer(d18:2/20:0) (see the structures below, which are included as non-limiting examples of ceramides that can be included in the beverage mix compositions provided herein). The nomenclature describes the structure of the ceramide molecule, including the number of hydroxyl groups, the sphingoid backbone composition, and the length and composition of the fatty acid side chain.

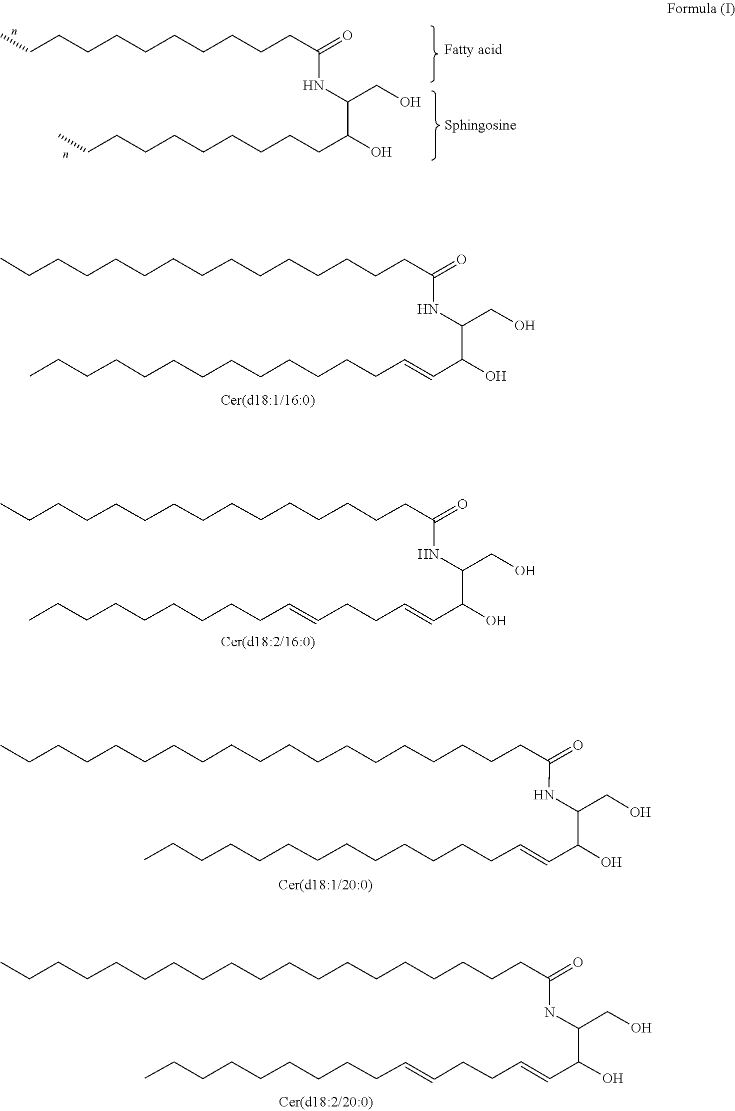

Ceramide compositions can be standardized to total sphingolipids, digalactosyl diglyceride (DGDG), or both. Ceramides can be obtained from any appropriate source (e.g., wheat, rice, soy, corn, nuts, eggs, milk, and/or seeds). For example, ceramides from wheat can contain about 1-4% glucosylceramide+ceramide and about 9-30% DGDG. As another example, ceramides from rice can contain about 6% glucosylceramide. In some cases, the compositions provided herein can include ceramides with about 40% DGDG and about 50% sphingolipids, which can provide a relatively high concentration of ceramides. In some cases, ceramides can be commercially obtained, and may be in powder or oil form.

Without being bound by theory, the combination of (1) bioactive collagen peptides, which can enhance the extracellular matrix by a protein-based mechanism, and (2) ceramides, which can enhance the stratum corneum layer by a fat-based mechanism, can synergize to support the skin, reduce wrinkles, and improve hydration.

Ceramides can be present in the beverage compositions provided herein in any appropriate amount. For example, from about 3 mg to about 300 mg (e.g., about 3 mg to about 10 mg, about 10 mg to about 50 mg, about 20 mg to about 40 mg, about 25 mg to about 100 mg, about 100 mg to about 300 mg, about 20 mg, about 25 mg, about 30 mg, about 35 mg, or about 40 mg) of the ceramides can be included in a beverage composition as described herein. In some cases, a beverage composition provided herein can contain about 0.02 wt % to about 2 wt % (e.g., about 0.05 wt % to about 0.5 wt %, about 0.1 wt % to about 1 wt %, about 0.2 wt % to about 0.3 wt %, about 0.2 wt %, about 0.25 wt %, about 0.27 wt %, about 0.3 wt %, or about 0.5 wt %) of ceramides.

### Vitamin A

Vitamin A is a fat-soluble polyunsaturated hydrocarbon vitamin that includes retinoids and carotenoids. Vitamin A obtained from plant sources is a carotenoid that the human body can transform into a retinol, while the vitamin A from animal sources is already in a form of retinol that is easily absorbed by the body. Palmitic acid is a 16-carbon saturated fatty acid, and is typically considered to be the most common saturated fatty acid found in animals, plants, and microorganisms. In some cases, the beverage compositions provided herein can contain vitamin A and/or vitamin A palmitate (VAP), which is the ester of retinol and palmitic acid, and can be converted into retinol and ultimately into retinoic acid. Vitamin A and/or VAP can be useful for their antioxidant activity, for example.

The degradation of VAP in aqueous solution is rapid, and oxygen can accelerate photo-catalyzed degradation of retinoids under light or chemically generated free radicals. In addition, the solubility of retinoids in aqueous solvents is poor because of their low polarity. Therefore, there is a need to deliver VAP in a form that protects it from chemical degradation during storage. Dispersibility and stability can be improved by encapsulating VAP oil in powder microcapsules by using suitable barrier components. Different techniques can be used to encapsulate VAP including, for example, spray drying of a formulated emulsion or suspension is a widely used technique for preparation of microcapsules.

For spray drying processes, carbohydrates, gums, semisynthetic cellulose derivatives, and synthetic polymers are commonly used carriers. Maltodextrin is a hydrolyzed starch having multiple functions, including film formation properties, binding ability, and reduction of oxygen permeability of the barrier component. Maltodextrin, however, has a low glass transition temperature and low emulsifying capacity to agglomeration or caking of micro particulate powders. Therefore, to increase the efficiency of microencapsulation, combinations of barrier materials can be used.

The powdered beverage compositions provided herein can contain any appropriate amount of vitamin A and/or VAR For example, vitamin A and/or VAP can be included in a composition provided herein in an amount from about 300 IU to about 6000 IU (e.g., about 500 IU to about 5000 IU, about 1000 IU to about 3000 IU, about 1250 IU to about 1750 IU, or about 1500 IU). In some cases, a beverage composition provided herein can contain about 0.001 wt % to about 0.5 wt % (e.g., about 0.01 wt % to about 0.1 wt %, about 0.05 wt % to about 0.15 wt %, about 0.1 wt % to about 0.5 wt %, about 0.05 wt %, about 0.07 wt %, about 0.1 wt %, or about 0.2 wt %) of VAP.

### Ascorbic Acid

Ascorbic acid is a chemical compound (C₆H₈O₆) commonly known as vitamin C. Vitamin C that is found in nature and can be used as an antioxidant additive in food and drink, as slowing oxidation can preserve color and freshness. The low pH of ascorbic acid also can help prevent microbial growth, thereby preventing or reducing spoilage and preserving freshness.

Ascorbic acid can be used in a variety of forms, including salts and esters. In such forms, ascorbic acid typically appears on ingredient lists under different names, such as sodium ascorbate, calcium ascorbate, potassium ascorbate, ascorbyl palmitate, or ascorbyl stearate. The powdered beverage compositions provided herein can include any appropriate amount of ascorbic acid. For example, ascorbic acid can be included in a composition provided herein in an amount from about 9 mg to about 450 mg (e.g., about 18 mg to about 360 mg, about 25 mg to about 180 mg, about 30 mg to about 60 mg, about 40 mg to about 100 mg, about 45 mg, about 55 mg, or about 90 mg). In some cases, a beverage composition provided herein can contain about 0.01 wt % to about 5 wt % (e.g., about 0.05 wt % to about 1 wt %, about 0.5 wt % to about 1.5 wt %, about 0.1 wt % to about 1 wt %, about 0.4 wt %, about 0.45 wt %, or about 0.5 wt %) of ascorbic acid.

### Astaxanthin

In some cases, a beverage composition provided herein can contain astaxanthin. Astaxanthin is an antioxidant with a unique size, shape, and physical properties that allow it to position itself across the cell membrane to protect the cell from free radical damage inside and out. Astaxanthin can help to protect cells from photoaging and UV-induced oxidative stress. The optional inclusion of astaxanthin in the compositions provided herein can reduce matrix metalloproteinase content, which can reduce the breakdown of skin collagen and elastin, allowing skin to maintain better firmness and hydration through improved barrier function and reduced water loss. Astaxanthin also can decrease sensitivity to UV exposure, reducing redness (versus placebo) after exposure to a fixed duration and intensity of light.

When astaxanthin is present in a beverage composition provided herein, the beverage composition can contain any appropriate amount of astaxanthin. For example, a beverage composition provided herein can contain from about 2 mg to about 12 mg (e.g., about 3 mg to about 8 mg, about 4 mg to about 9 mg, about 6 mg to about 8 mg, about 5 mg to about 10 mg, or about 6 mg to about 12 mg) of astaxanthin. In some cases, a beverage composition provided herein can contain about 0.05 wt % to about 10 wt % (e.g., about 0.1 wt % to about 5 wt %, about 0.5 wt % to about 2.5 wt %, about 1 wt % to about 2 wt %, about 0.5 wt %, about 1 wt %, about 1.5 wt %, or about 2 wt %) of astaxanthin. The astaxanthin can be natural astaxanthin, synthetic astaxanthin, or a combination thereof. When astaxanthin is included in a composition provided herein, the combination of the bioactive and standard collagen peptides and the optional astaxanthin can work to increase collagen production and reduce collagen breakdown, respectively. Further, the efficacy of the collagen peptides and the optional astaxanthin can be enhanced by additional ingredients or delivery technologies, as described herein.

### Additional Ingredients

In addition to the ingredients described above, the beverage compositions provided herein can contain any appropriate additional ingredients. For example, one or more additional ingredients can be included in the compositions provided herein to serve as, without limitation, emulsifiers, solubilizers, flavorings, colorings, stabilizers, sweeteners, and/or minerals. Representative ingredients that can be included in a composition as described herein include, for example, salts (e.g., sodium chloride) and/or silicon dioxide.

In some cases, one or more salts can be included in a beverage composition provided herein. Without being bound by theory, salts can act to enhance sweetness and balance bitterness. For example, salts can enhance citrus flavors and also can add depth and texture in carbonated beverages.

In some cases, silicon dioxide can be included in a beverage composition provided herein. Silicon dioxide is used in the food and beverage industries, both as an additive and as a flavoring intermediate. Silicon dioxide can help in the flow and mixing of substances during production, and can absorb moisture in raw materials and in the air to protect important ingredients such as medicines. Silicon dioxide also can be used as an anti-caking agent in powdered beverages such as milk powder, fruit juice powder, powdered drinks, and seasoning powder. Silicon dioxide can serve as an anti-caking agent because it can absorb water or bind very well to water, and may absorb water in an amount up to 2 times its own body weight. Silicon dioxide also can help to absorb water from the atmosphere that surrounds the products within packaging, and can help to absorb water from the surface of powdered products as well.

Other examples of ingredients that can be included in a beverage composition as described herein also include, without limitation, calcium (e.g., as calcium carbonate, dicalcium phosphate, calcium citrate, calcium gluconate, and/or a calcium amino acid polysaccharide complex), iron (e.g., as an iron amino acid polysaccharide complex), phosphorus (e.g., as dicalcium phosphate), iodine (e.g., as iodine amino acid polysaccharide complex), magnesium (e.g., as magnesium oxide, magnesium lactate, magnesium citrate, and/or a magnesium amino acid polysaccharide complex), zinc (e.g., as a zinc amino acid polysaccharide complex), selenium (e.g., as a selenium amino acid polysaccharide complex), copper (e.g., as a copper amino acid polysaccharide complex), manganese (e.g., as a manganese amino acid polysaccharide complex), chromium (e.g., as a chromium amino acid polysaccharide complex), molybdenum (e.g., as a molybdenum amino acid polysaccharide complex), boron (e.g., as a boron amino acid polysaccharide complex), vitamin D (e.g., as cholecalciferol), vitamin E (e.g., as d-alpha tocopheryl succinate), vitamin K (e.g., as phytonadione), thiamin (e.g., as thiamin HCl), riboflavin, niacin (e.g., as niacinamide), vitamin B6 (e.g., as pyridoxine HCl), folate (e.g., as folic acid or methyl folate), vitamin B12 (e.g., as cyanocobalamin or methylcobalamin), and combinations thereof.

Additional ingredients can be included in a beverage composition as described herein in any appropriate amount. For example, ingredients in addition to the collagen peptides, ceramides, vitamin A, vitamin C, astaxanthin (if included), and biotin (if included) can be present in a beverage composition provided herein in amounts ranging between trace amounts up to about 5 g (e.g., about 0.01 mg to about 1 g, about 0.1 mg to about 1 mg, about 0.5 mg to about 5 mg, about 5 mg to about 25 mg, or about 1 mg to about 100 mg).

### Methods of Making a Collagen and Ceramide Drink Composition

The beverage compositions described herein can be formulated for oral administration. In some embodiments, the compositions described herein can be formulated as a powdered beverage mix that can be mixed with a pharmaceutically acceptable aqueous carrier (e.g., an ingestible fluid) by the user. In some embodiments, the compositions described herein can be formulated as an already mixed beverage. Suitable aqueous carriers include, without limitation, water, alcohols, saline, and buffered solutions. Suitable aqueous carriers also can include physiologically acceptable aqueous vehicles (e.g., physiological saline) or other carriers appropriate for oral administration.

In some cases, the compositions provided herein can contain pharmaceutically acceptable additives. For example, a composition provided herein can contain one or more additives such as, without limitation, one or more suspending agents, emulsifying agents, non-aqueous vehicles, preservatives, buffer salts, flavoring agents, coloring agents, sweetening agents, suitable excipients, flavorings, colorants, and/or other ingredients as appropriate. For oral administration, one or more pharmaceutically acceptable excipients or carriers can include, for example, one or more binding agents, fillers, lubricants, disintegrants, and/or wetting agents.

In some cases, the collagen and ceramide-containing beverage compositions described herein can be mixed with an aqueous carrier (e.g., water) at room temperature, while in other cases, the collagen peptides described herein can be mixed with an aqueous carrier (e.g., water, coffee, cocoa, or tea) in the presence of heat (about 22° C. to about 82° C., about 22° C., about 27° C., about 32° C., about 37° C., about 42° C., about 47° C., about 47° C., about 52° C., about 57° C., about 62° C., about 67° C., about 72° C., about 77° C., about 82° C., about 22° C. to about 27° C., about 27° C. to about 32° C., about 32° C. to about 37° C., about 37° C. to about 42° C., about 42° C. to about 47° C., about 47° C. to about 52° C., about 52° C. to about 57° C., or about 57° C. to about 62° C., about 62° C. to about 67° C., about 67° C. to about 72° C., about 72° C. to about 77° C., or about 77° C. to about 82° C.). If heating is used, the mixture of collagen peptides and water can be heated for any appropriate length of time (e.g., about 5 minutes to about 30 minutes, about 5 minutes to about 10 minutes, about 10 minutes to about 15 minutes, about 15 minutes to about 20 minutes, about 20 minutes to about 25 minutes, or about 25 minutes to about 30 minutes).

The term “dose” or “total dose” refers to the amount of ingredient(s) administered (or to be administered) to a mammal (e.g., a human) over a designated period of time. For example, a “daily dose” would be understood to be the amount of ingredient(s) administered (or to be administered) over a 24 hour period of time in a suitable volume. A total daily dose can be prepared for administration in the form of one or more dosage units (e.g., one, two, three, or four beverages). In some cases, one or more dosage units can be administered in one or more dosages over the course of 24 hours (e.g., one dose, two doses, or three doses), where the total amount of ingredient(s) in the one or more dosages does not exceed the total daily dose.

In some cases, a serving can be at least 8 g of a beverage mix composition provided herein in powder form. In some cases, a serving of a beverage composition provided herein can be about 8 g to about 20 g, about 10 g to about 18 g, about 12 g to about 16 g about 12 g to about 14 g, about 10 g to about 14 g, about 10 g, about 12 g, or about 14 g, in powder form. In some cases, a serving of a beverage mix composition provided herein can be about 10 g to about 14 g in powder form. In some cases, a serving of a beverage mix composition provided herein can be about 12 g in powder form.

In some cases, one serving of a beverage mix composition provided herein in powder form can be dissolved into at least 8 fluid ounces of liquid (e.g., water or any other suitable beverage). In some cases, a serving of the beverage mix composition in powder form can be dissolved in about 8 fluid ounces to about 24 fluid ounces of liquid (e.g., about 10 fluid ounces to about 20 fluid ounces of liquid, about 12 fluid ounces to about 20 fluid ounces of liquid, about 14 fluid ounces to about 20 fluid ounces of liquid, about 14 fluid ounces to about 18 fluid ounces of liquid, or about 15 fluid ounces to about 17 fluid ounces of liquid). In some cases, a serving of a beverage mix composition provided herein in powder form can be dissolved in about 8 fluid ounces of liquid to about 20 fluid ounces of liquid. In some cases, a serving of a beverage mix composition provided herein in powder form can be dissolved in about 16 fluid ounces of liquid.

In some cases, a container or package containing a dry powder beverage mix composition provided herein can include a measuring scoop or spoon sized to measure out a single serving of a dry powder beverage mix composition provided herein. For example, a package (e.g., a jar, pouch, or any other suitable container) can contain multiple servings of a dry powder beverage mix composition provided herein, and can be provided to a subject in combination with a measuring spoon, a scoop, or any other device suitable for measuring out a single serving of a dry powder beverage mix composition provided herein. In some cases, a single serving of a dry powder beverage mix composition provided herein can be packaged in a single serving packet, pouch, or any other appropriate container from which the dry powder beverage mix composition can be dispensed into a fluid (e.g., water or another beverage) before consumption. In some cases, a plurality of such single serving containers can be packaged together (e.g., in a box or a larger pouch).

Methods of Improving Skin, Joint, Nail, and/or Hair Health

In some cases, the methods and compositions provided herein can be used to improve skin, joint and/or hair health in a mammal (e.g., a human). The methods can include administering to a mammal, or directing a mammal to self-administer, an effective amount of a composition provided herein, where the composition includes collagen peptides and ceramides as described herein. An effective amount of a composition can be, for example, a beneficial daily dose as set forth herein.

Skin health can be evaluated using any appropriate criteria. For example, a level of hydration, elasticity, firmness, smooth texture, even color, and/or a lack of blemishes (e.g., pimples, acne, and/or blackheads) can be used to evaluate the healthiness of skin.

Hair health can be evaluated using any appropriate criteria. For example, the health of hair can be evaluated based on the rate of growth, fullness, luster, sheen, the amount of breakage, and/or the amount of moisture.

In some embodiments, skin, joint and/or hair health can be determined before ingesting a composition described herein and then again after the composition has been ingested for a period of time (e.g., 2 weeks, one month, 6 months).

The invention will be further described in the following examples, which do not limit the scope of the methods and compositions of matter described in the claims.

### EXAMPLES

It would be understood that certain ingredients in the recipes shown below such as, without limitation, sodium chloride, are optional (i.e., may be removed from any of the recipes shown below).

Any of the suppliers and/or branded materials described herein may change; those listed in any of the Tables below are representative only.

### Example 1—First and Second Exemplary Collagen/Ceramide Drink Mixes

Tables 1A and 1B show representative recipes for beverage compositions as described herein.

TABLE 1A
First Exemplary Collagen/Ceramide Drink Mix
Ingredient
Collagen Hydrolysate
Bioactive Collagen Peptides
Ceramides
Vitamin A
Vitamin C
Sodium Chloride

TABLE 1B
Second Exemplary Collagen/Ceramide Drink Mix
Ingredient
Collagen Hydrolysate
Bioactive Collagen Peptides
Phytoceramide complex-Triticum aestivum L. (seed) extract
Vitamin A
Vitamin C
Sodium Chloride
Astaxanthin

### Example 2—Third and Fourth Exemplary Collagen/Ceramide Drink Mixes

Tables 2A and 2B show additional representative recipes for beverage compositions as described herein.

TABLE 2A
Third Exemplary Collagen/Ceramide Drink Mix

| | Amount per |
|---|---|
| Ingredient | serving |
| Collagen Hydrolysate | 5 g |
| Bioactive Collagen Peptides | 5 g |
| Phytoceramide complex- | 30 mg |
| Triticum aestivum L. (seed) extract | |
| Retinol palmitate | 450 mcg |
| Ascorbic acid | 45 mg |
| Sodium chloride | 70 mg |

TABLE 2B
Fourth Exemplary Collagen/Ceramide Drink Mix

| | Amount per |
|---|---|
| Ingredient | serving |
| Collagen Hydrolysate | 5 g |
| Bioactive Collagen Peptides | 5 g |
| Phytoceramide complex- | 30 mg |
| Triticum aestivum L. (seed) extract | |
| Retinol palmitate | 450 mcg |
| Ascorbic acid | 45 mg |
| Sodium chloride | 70 mg |
| Astaxanthin | 4 mg |

### Example 3—Fifth and Sixth Exemplar Collagen/Ceramide Drink Mixes

Tables 3A and 3B show additional representative recipes for beverage compositions as described herein.

TABLE 3A
Fifth Exemplary Collagen/Ceramide Drink Mix

| | Label Claim | Label |
|---|---|---|
| Ingredient | (UM/Serving) | UOM |
| Bioactive Collagen Peptides (2-5 kDa) | 5000 | mg |
| Bioactive Collagen Peptides (1-4 kDa) | 2500 | mg |
| Collagen Hydrolysate | 2500 | mg |
| Ceramide powder | 30 | mg |
| Ascorbic acid | 45 | mg |
| Sodium chloride | 100 | mg |
| Retinol Palmitate Vitamin A Spry Dry | 1500 | IU |
| Silicone dioxide | 75 | mg |

TABLE 3B
Sixth Exemplary Collagen/Ceramide Drink Mix

| | Label Claim | Label |
|---|---|---|
| Ingredient | (UM/Serving) | UOM |
| Bioactive Collagen Peptides (2-5 kDa) | 5000 | mg |
| Bioactive Collagen Peptides (1-4 kDa) | 2500 | mg |
| Collagen Hydrolysate | 2500 | mg |
| Ceramide powder | 30 | mg |
| Ascorbic acid | 45 | mg |
| Sodium chloride | 100 | mg |
| Retinol Palmitate Vitamin A Spry Dry | 1500 | IU |
| Silicone dioxide | 75 | mg |
| 2.5% Astaxanthin | 4 | mg |

### Example 4—Seventh and Eighth Exemplary Collagen/Ceramide Drink Mixes

Tables 4A and 4B show representative detailed recipes for beverage compositions as described herein.

TABLE 4A
Detailed Fourth Exemplary Collagen/Ceramide Drink Mix

| | Label Claim | Label | % | % | Input | Wt |
|---|---|---|---|---|---|---|
| Ingredient | (UM/Serving) | UOM | Activity | Overage | (mg) | (%) |
| Bioactive Collagen | 2500 | mg | 91 | 2 | 2802.20 | 20-30 |
| Peptides (1-4 kDa) | | | | | | |
| Bioactive Collagen | 5000 | mg | 91 | 2 | 5618.13 | 40-50 |
| Peptides (2-5 kDa) | | | | | | |
| Peptiplus ® Collagen | 2921.8 | mg | 91 | 2 | 3283.01 | 20-30 |
| AGGL | | | | | | |
| Ceramide powder | 30 | mg | 100% | 10% | 33.00 | 0.1-0.5 |
| Ascorbic acid | 45 | mg | 98 | 20 | 55.10 | 0.3-0.8 |
| Sodium chloride | 55 | mg | 44 | 0 | 125.00 | 0.5-1.5 |
| Retinol Palmitate | 1500 | IU | 25000 | 42 | 8.52 | 0.01-0.1 |
| Vitamin A Spry Dry | | | | | | |
| Silicon dioxide | 77.5 | mg | | | 77.50 | 0.1-1 |

TABLE 4B
Detailed Fifth Exemplary Collagen/Ceramide Drink Mix

| | Label Claim | Label | % | % | Input | Wt |
|---|---|---|---|---|---|---|
| Ingredient | (UM/Serving) | UOM | Activity | Overage | (mg) | (%) |
| Bioactive Collagen | 2500 | mg | 91 | 2 | 2802.20 | 20-30 |
| Peptides (1-4 kDa) | | | | | | |
| Bioactive Collagen | 5000 | mg | 91 | 2 | 5618.13 | 40-50 |
| Peptides (2-5 kDa) | | | | | | |
| Peptiplus ® Collagen | 2921.8 | mg | 91 | 2 | 3283.01 | 20-30 |
| AGGL | | | | | | |
| Ceramide powder | 30 | mg | 100% | 10% | 33.00 | 0.1-0.5 |
| Ascorbic acid | 45 | mg | 98 | 20 | 55.10 | 0.3-0.8 |
| Sodium chloride | 55 | mg | 44 | 0 | 125.00 | 0.5-1.5 |
| Retinol Palmitate | 1500 | IU | 25000 | 42 | 8.52 | 0.01-0.1 |
| Vitamin A Spry Dry | | | | | | |
| 2.5% Astaxanthin | 4 | mg | 2.5 | 20 | 192.00 | 0.5-2.5 |
| (water soluble) | | | | | | |
| Silicon dioxide | 77.5 | mg | | | 77.50 | 0.1-1 |

### Example 5—Ingesting a Collagen/Ceramide Drink Mix

A dry mix is relatively tasteless when mixed with a flavored drink product. Consumers ingesting a collagen beverage provided herein on a regular basis (e.g., daily for at least a few weeks (e.g., a month)) report improved mobility with the same or less pain than prior to starting the collagen/ceramide beverage regimen. Consumers ingesting a collagen/ceramide beverage regularly as described herein also report feeling less joint pain and fuller, thicker hair than prior to starting the collagen beverage regimen. In some instances, consumers report a reduction in hair loss and/or skin dryness.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A powdered beverage composition comprising first collagen peptides, second collagen peptides, and ceramides, wherein the first collagen peptides have an average molecular weight of about 1 kDa to about 4 kDa, and wherein the second collagen peptides have an average molecular weight of about 2 kDa to about 5 kDa.

2. The powdered beverage composition of claim 1, wherein the first collagen peptides are present in the composition in an amount of from about 15 wt % to about 30 wt %.

3. The powdered beverage composition of claim 1, wherein the second collagen peptides are present in the composition in an amount of from about 35 wt % to about 55 wt %.

4. The powdered beverage composition of claim 1, wherein the ceramides are present in the composition in an amount of from about 0.1 wt % to about 1 wt %.

5. The powdered beverage composition of claim 1, further comprising one or more vitamins.

6. The powdered beverage composition of claim 5, wherein the one or more vitamins comprise vitamin A palmitate, ascorbic acid, or vitamin A palmitate and ascorbic acid.

7. The powdered beverage composition of claim 6, wherein the composition comprises vitamin A palmitate in an amount of from about 0.01 wt % to about 0.1 wt %.

8. The powdered beverage composition of claim 6, wherein the composition comprises ascorbic acid in an amount of from about 0.1 wt % to about 1 wt %.

9. The powdered beverage composition of claim 1, further comprising astaxanthin.

10. The powdered beverage composition of claim 9, wherein the astaxanthin is present in the composition in an amount of from about 0.5 wt % to about 2.5 wt %.

11. The powdered beverage composition of claim 1, wherein the composition is contained in a bulk package, the bulk package containing a measuring scoop and the powdered beverage composition, the measuring scoop being sized to scoop an amount of the powdered beverage composition suitable for blending with a liquid to form a beverage, and wherein the amount of the powdered beverage composition is 10 to 20 g and comprises:
(a) from about 15 wt % to about 30 wt % of the first collagen peptides;
(b) from about 35 wt % to about 55 wt % of the second collagen peptides; and
(c) from about 0.1 wt % to about 0.5 wt % of the ceramides.

12. A powdered beverage composition comprising collagen peptides, ceramides, and at least one antioxidant, wherein the collagen peptides have an average molecular weight of about 1 kDa to about 5 kDa.

13. The powdered beverage composition of claim 12, wherein the collagen peptides comprise first collagen peptides and second collagen peptides, wherein the first collagen peptides have an average molecular weight of about 1 kDa to about 4 kDa, wherein the second collagen peptides have an average molecular weight of about 2 kDa to about 5 kDa, and wherein the ceramides are present in the composition in an amount of from about 0.1 wt % to about 1 wt %.

14. The powdered beverage composition of claim 12, wherein the at least one antioxidant is selected from vitamin A and vitamin C.

15. A powdered beverage composition comprising first collagen peptides, second collagen peptides, ceramides, and astaxanthin, wherein the first collagen peptides have an average molecular weight of about 1 kDa to about 4 kDa, wherein the second collagen peptides have an average molecular weight of about 2 kDa to about 5 kDa, and wherein the ceramides are present in the composition in an amount of from about 0.1 wt % to about 1 wt %.

16. A method of improving skin, joint, and/or hair health, where the method comprises:
solubilizing the powdered beverage composition of claim 1 in an aqueous carrier; and
ingesting an effective amount of the solubilized beverage composition, thereby improving skin, joint, and/or hair health.

17. The method of claim 16, wherein the aqueous carrier is water, coffee, cocoa, or tea.

18. A method of making the beverage mix of claim 1, wherein the method comprises combining the first collagen peptides, the second collagen peptides, and the ceramides.

19. The method of claim 18, further comprising adding an aqueous carrier.

20. The method of claim 19, wherein the aqueous carrier is water, coffee, cocoa, or tea.
